(19) United States
(12) Patent Application Publication
(10) Pub. No.: US 2020/0281575 A1
(43) Pub. Date: Sep. 10, 2020

(54) NEEDLE STABILIZATION SYSTEM AND METHODS OF USE

(71) Applicants: Gurvan Edmond Blackman, Robbinsville, NJ (US); David Christopher Feldstein, Haddonfield, NJ (US)

(72) Inventors: Gurvan Edmond Blackman, Robbinsville, NJ (US); David Christopher Feldstein, Haddonfield, NJ (US)

(21) Appl. No.: 16/809,253

(22) Filed: Mar. 4, 2020

Related U.S. Application Data
(60) Provisional application No. 62/813,227, filed on Mar. 4, 2019.

(51) Int. Cl. A61B 10/02; A61B 90/00; A61L 31/14

(52) U.S. Cl. CPC A61B 10/0266; A61B 2046/205; A61L 31/145; A61B 90/37

(57) ABSTRACT

A needle stabilizing device includes a first body having a substantially cylindrical portion and a spherical portion and defining a central lumen therethrough, a base having a concave seat to accept the spherical body of the first body, and a lateral extension, the base defining a secondary lumen in communication with the central lumen of the first body, the spherical portion and the seat of the base being movable with respect to one another with respect to at least two axes, and an adhesive layer coupled to the base and having an adhesive surface on an end opposite the base.

### CROSS-REFERENCE TO RELATED APPLICATIONS

This application claims priority to U.S. Provisional Application Ser. No. 62/813,227, filed Mar. 4, 2019, entitled “NEEDLE STABILIZATION SYSTEM AND METHODS OF USE,” the contents of which are fully incorporated as if fully set forth herein.

### FIELD OF THE DISCLOSURE

The present disclosure relates to needle stabilizers for use in medical settings. More particularly the present disclosure relates to temporary needle stabilizers for use during biopsies and other interventional procedures.

### BACKGROUND OF THE DISCLOSURE

Certain medical procedures employ the use of needles to puncture a patient's skin. The use of such needles is typically temporary. One example of such a procedure is a biopsy, in which a needle is passed to a target location within the body to sample tissue. After extraction, the sampled tissue is analyzed to determine if the tissue is benign, malignant or infectious. For example, biopsies may be used to diagnose lymphoma, kidney cancer, liver cancer, lung cancer, and other soft tissue cancer. A biopsy might also be needed to diagnose a variety of other conditions and/or diseases.

Because the tissue to be sampled may be hard to reach, and in locations that are not outwardly visible, the use of imaging systems is helpful in safely guiding the biopsy needle. These imaging systems may include computed tomography (CT), real-time X-ray (fluoroscopy), ultrasound, or other similar technologies, or combinations thereof.

Problems may occur during these procedures as the physician attempts to navigate the needle to the proper location for sampling. For example, biopsy needles may become displaced during the procedure, especially at times when the physician is reviewing the imaging systems, when the patient is entering the CT scanner, or if the patient inadvertently moves. In such cases, the physician may need to reintroduce/reposition the needle and/or relocate the position of the target tissue. This lengthens the procedure time and may increase the risk of procedural complications (e.g., bleeding, infection, damaging surrounding structures, etc.) and causes discomfort to the patient. Additionally, the physician is typically limited by having to hold the biopsy needle throughout the procedure.

### SUMMARY OF THE DISCLOSURE

In some embodiments, a needle stabilizing device includes a first body having a substantially cylindrical portion and a spherical portion and defining a central lumen therethrough, a base having a concave seat to accept the spherical body of the first body, and a lateral extension, the base defining a secondary lumen in communication with the central lumen of the first body, the spherical portion and the seat of the base being movable with respect to one another with respect to at least two axes, and an adhesive layer coupled to the base and having an adhesive surface on an end opposite the base.

In some embodiments, a needle stabilizing device includes a pad at least partially formed of a hydrogel having a density of between 0.9 g/mL and 0.9 g/mL and a height of at least 2 centimeters, and an adhesive material coupled to the pad.

In some embodiments, a method of stabilizing a biopsy needle includes providing a stabilizing device having a pad at least partially formed of a hydrogel having a density of between 0.9 g/mL and 2 g/mL and a height of at least 2 centimeters, and an adhesive material coupled to the pad, adhering the adhesive material to the patient's tissue to secure the pad to the patient's tissue, piercing the pad with the biopsy needle, and reviewing an imaging device to determine a position of the biopsy needle with respect to adjacent body tissue.

In some embodiments, a needle stabilizing device includes a hollow frustoconical body having a first circumference adjacent a first end of between 0.5 mm and 4 mm, and a second circumference adjacent a second end of between 6 cm and 16 cm, and defining a central opening centered around a longitudinal axis, and a ring-shaped adhesive material coupled to the frustoconical body on an end opposite the central opening, the adhesive material being ring shaped and having an opening to allow a needle to pass from the central opening to a patient's body tissue.

In some embodiments, a method of stabilizing a biopsy needle includes providing a stabilizing device comprising a hollow frustoconical body having a first circumference adjacent a first end of between 0.5 mm and 4 mm, and a second circumference adjacent a second end of between 6 cm and 16 cm, and defining a central opening centered around a longitudinal axis, and a ring-shaped adhesive material coupled to the frustoconical body on an end opposite the central opening, the adhesive material being ring shaped and having an opening to allow a needle to pass from the central opening to a patient's body tissue, adhering the adhesive material to the patient's tissue to secure the frustoconical body to the patient's tissue, passing a biopsy needle through the central opening, through the hollow frustoconical body along the longitudinal axis, and piercing the patient's body tissue, and reviewing an imaging device to determine a position of the biopsy needle with respect to adjacent body tissue.

In some embodiments, a needle stabilizing device includes a first body having a substantially cylindrical portion and a spherical portion and defining a central lumen therethrough, a base having a concave seat to accept the spherical body of the first body, and a lateral extension, the base defining a secondary lumen in communication with the central lumen of the first body, the spherical portion and the seat of the base being movable with respect to one another with respect to at least two axes, and an adhesive layer coupled to the base and having an adhesive surface on an end opposite the base.

In some embodiments, a method of stabilizing a biopsy needle includes providing a stabilizing device including a first body having a substantially cylindrical portion and a spherical portion and defining a central lumen therethrough, a base having a concave seat to accept the spherical body of the first body, and a lateral extension, the base defining a secondary lumen in communication with the central lumen of the first body, the spherical portion and the seat of the base being movable with respect to one another with respect to at least two axes, and an adhesive layer coupled to the base and having an adhesive surface on an end opposite the base, adhering the adhesive layer to the patient's tissue to secure the base to the patient's tissue, passing a biopsy needle through the central lumen of the first body, and the secondary lumen of the base, and piercing the patient's body tissue with the biopsy needle, reviewing an imaging device to determine a position of the biopsy needle with respect to adjacent body tissue, and adjusting the orientation of the cylindrical portion with respect to the base.

### BRIEF DESCRIPTION OF THE DISCLOSURE

Various embodiments of the presently disclosed needle stabilizers are shown herein with reference to the drawings, wherein:

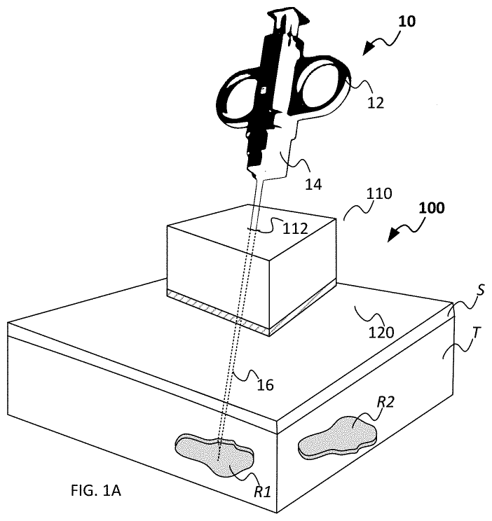

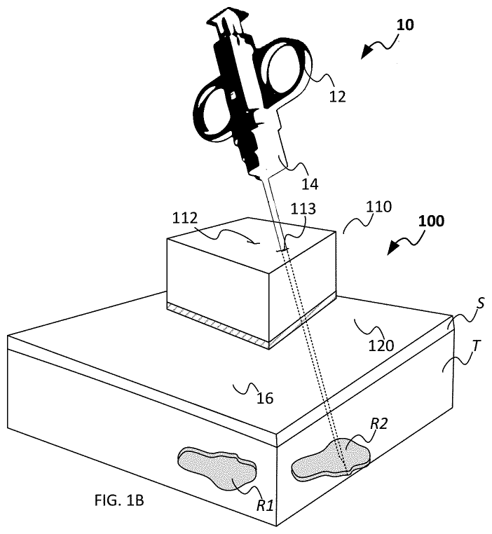

FIG. 1A is a schematic perspective view of a biopsy needle being inserted through a pad-like needle stabilizer according to one embodiment of the present disclosure;

FIG. 1B is a schematic perspective view of the biopsy needle and needle stabilizer of FIG. 1A after the location of the needle has been adjusted;

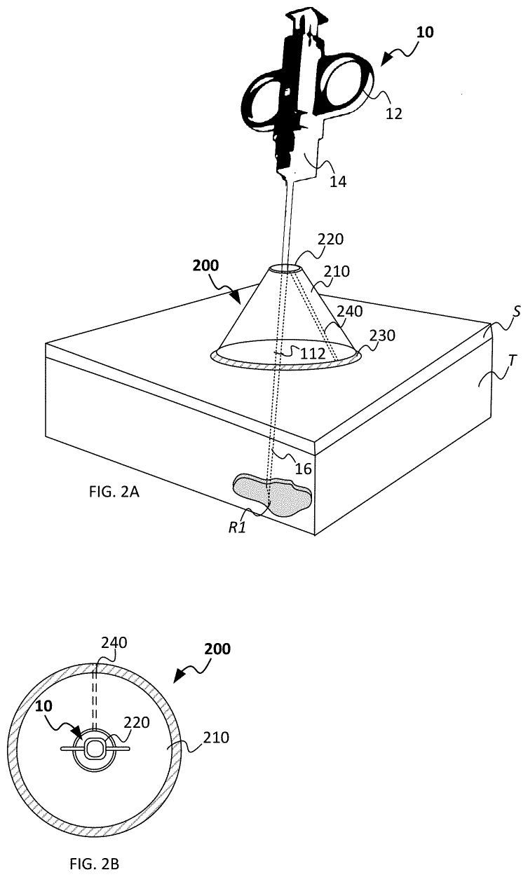

FIG. 2A is a schematic perspective view of a biopsy needle being inserted through a frustoconical needle stabilizer according to another embodiment of the present disclosure;

FIG. 2B is schematic top view of the frustoconical needle stabilizer of FIG. 2A;

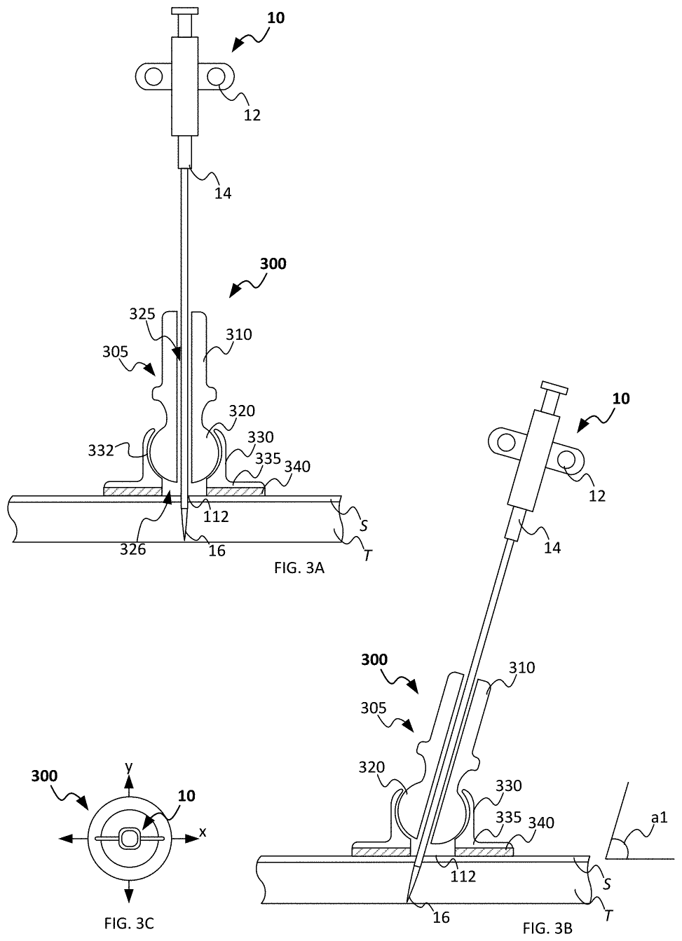

FIG. 3A is a schematic cross-sectional view of a ball-and-socket needle stabilizer according to a third embodiment of the present disclosure;

FIG. 3B is a schematic perspective view of the biopsy needle and needle stabilizer of FIG. 3A after the location of the needle has been adjusted; and

FIG. 3C is schematic top view of a biopsy needle disposed within the needle stabilizer of FIG. 3A.

Various embodiments of the present invention will now be described with reference to the appended drawings. It is to be appreciated that these drawings depict only some embodiments of the invention and are therefore not to be considered limiting of its scope.

### DETAILED DESCRIPTION

Despite the various improvements that have been made to biopsy needles and their methods of use, conventional devices suffer from some shortcomings as described above.

There therefore is a need for further improvements to the devices, systems, and methods of securing and stabilizing needles, such as biopsy needles. Among other advantages, the present disclosure may address one or more of these needs.

As used herein, the term “proximal,” when used in connection with a component of a needle, refers to the end of the component closest to the physician when the needle is inserted in a patient, whereas the term “distal,” when used in connection with a component of a needle assembly, refers to the end of the component farthest from the physician when the needle is inserted in a patient.

Likewise, the terms “trailing” and “leading” are to be taken as relative to the operator (e.g., physician) of the needle or biopsy assembly. “Trailing” is to be understood as relatively close to the operator, and “leading” is to be understood as relatively farther away from the operator.

The present disclosure relates to a needle stabilization system that lowers the possibility of displacement or accidental movement of a biopsy needle during a procedure. The needle stabilization system may also shorten procedure time, and improve patient comfort and safety by assisting the physician to accurately and quickly reach the target area of the biopsy, and maintaining the biopsy needle in place without the physician's hands.

FIG. 1A shows a conventional biopsy device 10 having a handle 12, a body 14 and a needle 16 having a compartment for obtaining a tissue sample. Several variations of biopsy devices are available but almost all include a needle with an opening capable of collecting a sample. As used herein, the terms “biopsy device,” and “biopsy needle” are used to refer to any such device having a needle for piercing the skin. Additionally, it will be understood that others needles, catheters, tubes, introducers, rods, shafts, screws and similar devices may be used in connection with some of these embodiments to increase stability. As shown, biopsy device 10 is being used in connection with one embodiment of the stabilizer according to the present disclosure. Specifically, needle stabilizer 100 includes a pad 110 having an adhesive layer 120 coupled thereto, the adhesive layer 120 being coupleable to a patient's skin “S” near the target tissue “T” disposed below the skin and having suspicious regions “R1” and “R2,” which are to be examined.

Pad 110 may be substantially cube-shaped as shown having a length, width and height of between 2 centimeters and 10 centimeters. Alternatively, pad 110 may be formed as a cylinder or disk having a diameter of between 2 centimeters and 10 centimeters, and a height of between 1 centimeter and 5 centimeters, or in other suitable shapes. Pad 110 may be formed of an aqueous material or suitable hydrogel such as those used in standoff pads common in ultrasonic imaging. Alternatively, pad 110 may be formed of a foam. In at least some examples, the pad 110 is translucent, bacteriostatic and/or hypoallergenic. In at least some example, pad 110 has a sufficient density that enables it to stabilize the biopsy device. For example, pad 110 may have a density of between 0.9 g/mL and 2 g/mL. It will be understood that a relationship exists between the density of the material and the height of the pad, and that a less dense pad may require a greater height to provide the requisite stability.

Adhesive layer 120 may be coupled to the bottom portion of pad 110 (i.e., the portion of the pad that is closest to the skin), and may have a lower surface opposite the pad to secure and adhere the pad 110 to a predetermined location on the patient's skin. Suitable materials for adhesive layer 120 may include acrylate, including methacrylates and/or epoxy diacrylates, rosin, copal and/or silica, and suitable combinations thereof.

With the pad 110 and adhesive layer 120 coupled over the target tissue, the user may introduce biopsy device 10 through pad 110 at entrance point 112. As shown, biopsy device 10, and specifically needle 16 of the device may be inserted through pad 110 and may begin to puncture the patient's skin “S”. With the biopsy device 10 inserted through the entire height of the pad 110, the physician may step away and examine the imaging system to determine if the needle approach is correct. In some embodiments, the needle may be stabilized within the biopsy device and secured by the device before puncturing the skin.

Specifically, once the device 10 has passed through pad 110, the pad may support biopsy device 10 and maintain its location and orientation, enabling the physician to leave the device 10 and turn his attention to the guidance system. After reviewing the position and orientation of biopsy device 10 and specifically the needle, the physician may (1) determine that the location of the needle is proper and that the target tissue is adjacent to needle 16, and begin to collect a sample; (2) continue to advance the biopsy device 10 along the same trajectory to reach the target tissue, (3) adjust the orientation and/or trajectory of the needle 16; or (4) completely remove the biopsy device 10 and begin the procedure at a new location 113 as shown in FIG. 1B. Thus, the process of inserting and adjusting the location of the needle, and comparing the location to the imaging system can be done and the physician may remove his hands from the biopsy needle when he is reviewing the imaging system, speaking to the patient or performing a different function without being concerned with the biopsy needle falling out, or losing the current position of the needle.

In another embodiment, a frustoconical stabilizing device 200 is used to secure the biopsy device 10. As shown in FIG. 2A, stabilizing device 200 includes a frustoconical body 210 having a general cone shape, and a central aperture 220 sized to receive a portion of biopsy device 10. Body 210 may be formed partially or entirely from a polymeric material, such as silicone, thermoplastic polyurethanes (TPU), rubber, metal, plastic, polypropylene, polyethylene, acrylonitrile butadiene styrene (ABS), high impact polystyrene (HIPS), polyvinyl chloride (PVC) polycarbonate, thermoplastic elastomers, polybutylene terephthalate, ethylene vinyl acetate, nylon a low-density polyethylene, linear low-density polyethylene, and suitable combinations thereof. First body 305 may also be formed at least partially or entirely of a metal such a stainless steel, titanium, or other biocompatible metal, or of wood or cardboard.

In at least some examples, central aperture 220 has a diameter of between 0.5 millimeters and 4 millimeters and may be sized to accept a biopsy needle having sizes of 10-25 gauge. An adhesive ring 230 is coupled to the other side of frustoconical body 210, opposite central aperture 220. Adhesive ring 230 may have a circumference of between 6 centimeters and 16 centimeters, and may include a glue or adhesive material such as those described above with respect to the embodiment of FIGS. 1A and 1B. Body 210 may include a scored region or line 240 extending from the central aperture along the body 210 to the adhesive ring 230 (FIG. 2B). A physician may split the body along scored line 240 after the procedure for easy removal of the stabilizing device.

Biopsy device 10 may be inserted through central aperture 220 into the interior of body 210, and down to the patient's skin. Biopsy device 10 may then puncture the skin at location 112 toward the target tissue. If the physician wishes to temporarily release the device 10, they may do so and the device will stay upright and/or in place as a portion of the biopsy device rests on a portion of the circumference of central aperture 220. As shown, central aperture 220 provides enough spacing so that the physician is capable of finely adjusting the approach angle of the needle with respect to target tissue without having to reposition the stabilizing device.

A third embodiment of a stabilizing device 300 is shown in FIGS. 3A-C. Stabilizing device 300 includes a first body 305 having a substantially cylindrical portion 310 terminating in a substantially spherical portion 320. Stabilizing device 300 may further include a second body in the form of a base 330 having a cup-like concavity 332 capable of accepting spherical portion 320 so that the spherical portion is securely seated therein. Base 330 further includes a lateral portion 335 that extends radially outward away from concavity 332. In at least some examples, spherical portion and concavity 332 are configured to have substantially smooth surfaces so that they can be moved relative to each other without much friction. In at least some examples, spherical portion and concavity 332 have a coefficient of static friction of between 0.1 and 0.9.

First body 305 may formed of any of the materials described above with respect to device 200, and base 330 may be formed of the same material or a different material as first body 305, and may be selected from any of the materials disclosed with respect to the first body 305.

First body 305 may be moveable relative to base 330. Specifically, spherical portion 320 and concavity 332 function as a ball-and-socket joint having three degrees of freedom, so that the spherical portion 320 and with it the cylindrical portion 310 can be oriented as desired. In at least some examples, the joint between the first body and the base is capable of only two degrees of freedom (e.g., the cylindrical portion is not capable of rotation about its central axis).

A central lumen 325 is defined through cylindrical portion 310 and spherical portion 320, and a secondary lumen 326 is defined within base 330. Central lumen 325 may have a diameter of between 0.5 millimeters and 4 millimeters, and secondary lumen 326 may have a diameter of between 2 centimeters and 16 centimeters. Central lumen 325 and secondary lumen 326 may be in communication so that a needle that is passed through central lumen may continue through secondary lumen 326 to reach the patient's skin “S”. In at least some examples, secondary lumen 326 has a larger diameter than central lumen 325 so that when the angle a1 of the cylindrical portion is changed (FIG. 3B), the needle is capable of easily passing through the secondary lumen 326.

Base 330 is secured to adhesive layer 340, which is configured to adhere to the skin in similar ways and using any of the materials described above with respect to the previous two embodiments.

In use, the physician may secure the adhesive layer 340 to the patient's skin over the target area and introduce the needle of the biopsy device into the central lumen of the cylindrical portion, through the spherical portion 320 and the secondary lumen of the base to pierce the patient's skin. If the physician desires to step away from the procedure, for example, to review the imaging system, he may leave the biopsy device to be supported by the cylindrical portion and return to it at a later point in time. If, after review of the imaging system, the physician decides to further advance the needle, he may do so. If, instead, the physician decides to change the trajectory of the needle, the spherical portion may be moved within the cup-like concavity of the base to adjust the angle of the cylindrical portion with respect to the skin. This adjustment may be made with respect to axis x or axis y as shown in FIG. 3C.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A method of stabilizing a biopsy needle comprising:
providing a stabilizing device having a pad at least partially formed of a hydrogel having a density of between 0.9 g/mL and 2 g/mL and a height of at least 2 centimeters, and an adhesive material coupled to the pad;
adhering the adhesive material to the patient's tissue to secure the pad to the patient's tissue;
piercing the pad with the biopsy needle; and
reviewing an imaging device to determine a position of the biopsy needle with respect to adjacent body tissue.

2. A method of stabilizing a biopsy needle comprising:
providing a stabilizing device comprising a hollow frustoconical body having a first circumference adjacent a first end of between 0.5 mm and 4 mm, and a second circumference adjacent a second end of between 6 cm and 16 cm, and defining a central opening centered around a longitudinal axis, and a ring-shaped adhesive material coupled to the frustoconical body on an end opposite the central opening, the adhesive material being ring shaped and having an opening to allow a needle to pass from the central opening to a patient's body tissue;
adhering the adhesive material to the patient's tissue to secure the frustoconical body to the patient's tissue;
passing a biopsy needle through the central opening, through the hollow frustoconical body along the longitudinal axis, and piercing the patient's body tissue; and
reviewing an imaging device to determine a position of the biopsy needle with respect to adjacent body tissue.

3. A method of stabilizing a biopsy needle comprising:
providing a stabilizing device including a first body having a substantially cylindrical portion and a spherical portion and defining a central lumen therethrough, a base having a concave seat to accept the spherical body of the first body, and a lateral extension, the base defining a secondary lumen in communication with the central lumen of the first body, the spherical portion and the seat of the base being movable with respect to one another with respect to at least two axes, and an adhesive layer coupled to the base and having an adhesive surface on an end opposite the base;
adhering the adhesive layer to the patient's tissue to secure the base to the patient's tissue;
passing a biopsy needle through the central lumen of the first body, and the secondary lumen of the base, and piercing the patient's body tissue with the biopsy needle;
reviewing an imaging device to determine a position of the biopsy needle with respect to adjacent body tissue; and
adjusting the orientation of the cylindrical portion with respect to the base.
